(19) United States
(12) Patent Application Publication
(10) Pub. No.: US 2015/0374649 A1
(43) Pub. Date: Dec. 31, 2015

(54) Treatment of Severe Hypertriglyceridemia

(71) Applicant: CymaBay Therapeutics, Inc, Newark, CA (US)

(72) Inventors: Pol Boudes, Fremont, CA (US); Yun-Jung Choi, Fremont, CA (US); Robert L. Martin, San Ramon, CA (US); Charles A. McWherter, Oakland, CA (US)

(73) Assignee: CymaBay Therapeutics, Inc, Newark, CA (US)

(21) Appl. No.: 14/749,934

(22) Filed: Jun. 25, 2015

Related U.S. Application Data
(60) Provisional application No. 62/017,444, filed on Jun. 26, 2014.

(51) Int. Cl. A61K 31/192; A61K 31/202; A61K 31/4406

(52) U.S. Cl. CPC A61K 31/192; A61K 31/4406; A61K 31/202

(57) ABSTRACT

Treatment of severe hypertriglyceridemia, such as Type I or Type V hyperlipoproteinemia, by therapy with MBX-8025 or an MBX-8025 salt, alone or in combination with one or more of a fibrate, niacin, and an omega-3 fatty acid, optionally accompanied by apheresis.

### CROSS-REFERENCE TO RELATED APPLICATION

This application claims the priority under 35 USC 119(e) of U.S. Application No. 62/017,444, filed Jun. 26, 2014, entitled “Treatment of severe hypertriglyceridemia”, which is incorporated into this application by reference.

### BACKGROUND OF THE INVENTION

1. Field of the Invention

This invention relates to the treatment of severe hypertriglyceridemia.

2. Description of the Related Art

Severe Hypertriglyceridemia

Dyslipidemia is the presence of an abnormal amount of lipids (e.g. cholesterol and/or fat) in the blood. In developed countries, most dyslipidemias are hyperlipidemias; that is, an elevation of lipids/lipoproteins in the blood—the term hyperlipidemia is often used to include hyperlipoproteinemia. Hyperlipidemias include hypercholesterolemia (elevated cholesterol) and hyperglyceridemia (elevated glycerides), with hypertriglyceridemia (HTG, elevated triglycerides (TGs)) as a subset of hyperglyceridemia. Berglund et al., “Evaluation and Treatment of Hypertriglyceridemia: An Endocrine Society Clinical Practice Guideline”, J. Clin. Endocrinol. Metab., 97(9), 2969-2989 (2012) define severe hypertriglyceridemia (SHTG) as referring to a serum TG level of more than 1000 mg/dL, and very severe hypertriglyceridemia as referring to a serum TG level of >2000 mg/dL. However, the “Third Report of the National Cholesterol Education Program (NCEP) Expert Panel on Detection, Evaluation, and Treatment of High Blood Cholesterol in Adults (Adult Treatment Panel III) Final Report”, Circulation, 106, 3143-3422 (2002) (NCEP ATP III), defines serum TG levels as “very high” at >500 mg/dL; and this value has been used to effectively define SHTG in the development of prescription omega-3 fatty acids: for example, EPANOVA, LOVAZA, and VASCEPA all are indicated for, as an adjunct to diet, reducing TG levels in adult patients with “severe (≧500 mg/dL) hypertriglyceridemia”.

Hypertriglyceridemia may be caused by one or both of genetically based disorders (primary disorders) and disorders caused by other diseases (secondary disorders). According to Ewald et al., “Treatment options for severe hypertriglyceridemia (SHTG): the role of apheresis”, Clin. Res. Cardiol. Suppl., 7, 31-35 (2012), the genetically well-characterized types of SHTG are those associated with familial lipoprotein lipase (LPL) deficiency and familial apolipoprotein C-II deficiency, which usually present in infancy as chylomicronemia syndromes causing SHTG in very early childhood. In adults, SHTG is usually associated with very high fasting levels of chylomicrons and very low density lipoprotein (VLDL), both of which serve as carriers for large quantities of TGs; and is probably of plurigenetic origins compounded by environmental and lifestyle factors. Gotanda et al., “Diagnosis and Management of Type I and Type V Hyperlipoproteinemia”, J. Atheroscler. Thromb., 19, 1-12 (2012), state that according to the WHO (Frederickson) classification of hyperlipoproteinemia, Type I hyperlipoproteinemia is characterized by an increase in chylomicrons alone, shows the severest HTG, and is classically represented by familial LPL deficiency and apolipoprotein C-II deficiency; while Type V hyperlipoproteinemia is characterized by an increase in both chylomicrons and VLDL. Type I hyperlipoproteinemia, also called familial chylomicron syndrome (FCS), is characterized by a marked elevation in plasma TGs after an overnight fast, and results from failure to properly metabolize and clear chylomicrons from the circulation. The most common defect in FCS is a deficiency of LPL resulting from a genetic mutation producing a loss of function in the LPL gene. In LPL deficiency, the severity of the HTG is related to the amount of ingested fats. In most patients, the disorder is diagnosed in childhood following repeated episodes of abdominal pain associated with pancreatitis and the presence of eruptive xanthomas. Type V hyperlipoproteinemia is characterized by a usually undefined deficit in the LPL system, and the clinical presentation is similar to that of Type I, except that Type V invariably presents in adulthood. Type V is also associated with a number of abnormalities that are known to make the patient more susceptible to cardiovascular disease, whereas Type I is not. There are many secondary causes of SHTG, including obesity, untreated diabetes mellitus, alcohol overconsumption, pregnancy, and the use of some drugs; while several of the secondary causes are associated with abnormalities of insulin responsiveness.

SHTG is well known to be associated with both cardiovascular disease and acute pancreatitis. The role of TGs in promoting cardiovascular disease were first postulated more than 65 years ago, and, according to Ewald et al., recent data on SHTG have established a consistently strong relationship between TG levels and cardiovascular risk. According to both Ewald et al. and Pejic et al, “Hypertriglyceridemia”, J. Am. Bd. Fam. Med., 19, 310-316 (2006), the role of SHTG in acute pancreatitis is well established, and the literature describes SHTG as the third most common cause for acute pancreatitis, after gallstones and alcohol. SHTG has been reported to account for up to 10% of all episodes of acute pancreatitis, and some studies on gestational pancreatitis even report SHTG as the underlying etiology in more than one-half of all cases; while there is even some evidence that hypertriglyceridemic pancreatitis is associated with higher severity and a higher complication rate. It is generally believed that TG levels above 10 mM (886 mg/dL) or 1000 mg/dL (physicians in different countries cite slightly different values based on the units that they conventionally use to measure TG levels) may trigger acute pancreatitis and its complications, and that TG levels above 20 mM (1772 mg/dL) or 2000 mg/dL are associated with the greatest risk, but the threshold is somewhat arbitrary and the level above which acute pancreatitis may occur is unknown; therefore, rapid lowering of very elevated serum TG levels at least to less than 1000 mg/dL, and preferably below 10 mM, is a primary medical goal in preventing serious harm to the patient with SHTG.

Treatments for Severe Hypertriglyceridemia

Lifestyle changes and dietary modifications are essential features in the management of SHTG: appropriate nutrition (lowering dietary fats and simple sugars), avoiding alcohol consumption, weight reduction, exercise, control of potential concomitant endocrinopathies (e.g. diabetes), and avoidance of drugs with hypertriglyceridemic side effects are critical. Other treatments, such as pharmacological treatments and apheresis, are generally adjuncts to these lifestyle changes and dietary modifications; thus in general treatments of the type disclosed and claimed in this application will be applied to persons who are already undertaking these lifestyle changes and dietary modifications, and such changes/modifications will not be expressly mentioned further.

Three common pharmacological treatments for hypertriglyceridemia and SHTG are fibrates, niacin, and omega-3 fatty acids.

Fibrates are derivatives of fibric acid (2-methyl-2-phenoxypropionic acid), and are the mainstay of hypertriglyceridemia treatment. They are agonists of peroxisome proliferator activated receptor-α (PPARα), increasing the activity of LPL, which causes a decrease in TG levels. According to Berglund et al. and Yuan et al., “Hypertriglyceridemia: its etiology, effects and treatment”, Can. Med. Assoc. J., 176(8), 1113-1120 (2007), fibrates raise high density lipoprotein cholesterol (HDL-C), and they may increase low density lipoprotein cholesterol (LDL-C), particularly if TG levels exceed 400 mg/dL, increasing the size and decreasing the density of the LDL-C particles. Five fibrates are used clinically: three are available in the United States: gemfibrozil, fenofibrate, and choline fenofibrate (the choline salt of fenofibric acid); the other two agents, bezafibrate and ciprofibrate, are available in Europe and elsewhere but not currently in the US. Clofibrate was formerly used, but was withdrawn some years ago for side effects. Fibrates can reduce serum TG levels by up to 50%, though there is a slow onset of TG lowering. The effectiveness of fibrates in reducing cardiovascular disease outcomes is of concern: while earlier studies showed that fibrates reduced cardiovascular event rates (e.g. gemfibrozil resulted in a statistically significant benefit in men with high TG and low HDL-C readings), the FIELD study reported in 2007 that plasma TG, LDL-C, and HDL-C levels in diabetic patients responded favorably to fenofibrate treatment, but the reduction in the primary endpoint of cardiovascular disease (16%) was not statistically significant, though secondary and tertiary outcomes were significantly improved. Fibrate therapy is generally well-tolerated, with rare reports of hepatitis or myositis.

Niacin (nicotinic acid, pyridine-3-carboxylic acid, vitamin B3) was first described as having lipid-lowering properties in 1955. According to Berglund et al. and Yuan et al., high-dose niacin (at least 1500 mg/day) decreases TG levels by at least 40% although, as with fibrates, there is a slow onset of TG lowering; and niacin can also raise HDL-C levels by 40% or more. Niacin also reliably and significantly lowers LDL-C levels, which the other major TG-lowering medications do not. In the Coronary Drug Project, niacin, in comparison with placebo, reduced coronary events. Niacin has multiple adverse effects, the worst of which is hepatitis. However, at doses of 1.5-2 g/d, complications are unusual. Sustained-release niacin is more hepatotoxic than immediate-release niacin but is better tolerated. Flushing, itching, and rash are expected adverse effects that are less common with long-acting formulations. These symptoms are an annoyance, and negatively affect compliance, but are not life threatening and may be minimized by starting at low doses and increasing slowly. Switching from immediate-release niacin to an equal dose of time-release preparation has been reported to cause severe hepatotoxicity. If niacin is prescribed for patients with type 2 diabetes, glucose control should be carefully monitored as modest increases in insulin resistance can occur. Also, niacin can increase blood levels of uric acid by blocking its excretion, and may precipitate or worsen gout.

Omega-3 fatty acids (O3FA, also called ω-3 fatty acids or n-3 fatty acids) are polyunsaturated fatty acids with a double bond at the third carbon atom from the end of the carbon chain opposite the carboxyl group. The three types of O3FA involved in human physiology are α-linolenic acid (ALA, found in plant oils), eicosapentaenoic acid (EPA), and docosahexaenoic acid (DHA), the latter two both being commonly found in marine oils. Common sources of animal omega-3 EPA and DHA fatty acids include fish oils (usually from fatty fish such as anchovy, mackerel, and sardines), egg oil, squid oils and hill oil. According to the US National Institute of Health's on-line information “Omega-3 Fatty Acids and Health: Fact Sheet for Health Professionals” (http://ods.od.nih.gov/factsheets/Omega3FattyAcidsandHealth-HealthProfessional/), O3FA are essential fatty acids, i.e. not synthesizable by the human body, though humans have a limited ability to convert ALA to EPA and an even more limited ability to convert EPA to DHA. Also, according to the Fact Sheet, from a review of a review of 123 articles in 2004 and 2005 strong evidence showed that fish-oil supplements had a substantial and beneficial effect on triglycerides that was greater with larger intakes of fish oil; most studies reported a net decrease of about 10-33%. Increasing the consumption of O3FA is considered a standard part of treatment of hypertriglyceridemia. In addition to over-the-counter supplements containing O3FA, there are three prescription products in the US: EPANOVA (a mixture of concentrated O3FA purified from crude fish oil containing EPA and DHA in their free fatty acid form at a total concentration of 50-60% EPA and 15-25% DHA in a gel capsule designed to release them in the ileum), LOVAZA (a gel capsule containing ethyl esters of O3FA sourced from fish oils, approximately 52% EPA ethyl ester and 42% DHA ethyl ester), and VASCEPA (a gel capsule containing EPA ethyl ester derived from fish oil). The term “an omega-3 fatty acid” or “O3FA” is used here to include both the free acids, particularly EPA or DHA, or a combination of omega-3 fatty acids, such as are derived from fish oils (as in EPANOVA and over-the-counter supplements), and also their esters (e.g. ethyl esters, as in LOVAZA and VASCEPA).

No drugs have been shown to be effective in the treatment of Type I hyperlipoproteinemia (LPL deficiency); and, despite the available therapies described above, including an appropriate (low-fat) diet, some patients with SHTG, including especially those with Type I or Type V hyperlipoproteinemia, remain refractory (that is, they fail to reach a TG level less than 1000 mg/dL, such as less than 10 mM, despite dietary changes and one or more of the therapies mentioned above).

A gene-replacement therapy for LPL deficiency, alipogene tiparvovec (GLYBERA), has been developed and was approved in Europe in 2012 (as an orphan drug), but has not yet been approved in the US. It is administered as a single treatment consisting of multiple injections into the leg muscles of 10¹² genome copies of the gene in a virus protein shell—47 injections for a 70 Kg patient: spinal or regional anesthesia (or deep sedation), is recommended for the procedure, and methylprednisolone pretreatment is required; and an immunosuppressive regimen is required for 3 days before and 12 weeks after the treatment. Pradigastat, an oral diacylgylcol acyltransferase-1 inhibitor, is currently in Phase 3 trials for FCS. CAT-2003, a conjugate of niacin with EPA, has completed three pilot Phase 2 trials in patients with HTG, including Type I hyperlipoproteinemia.

Apheresis is the removal of TGs and TG-rich lipoproteins from blood by therapeutic plasma exchange (TPE) or filtration. In TPE, blood is removed from the patient and the plasma separated, with cellular components being returned to the patient together with replacement fluid for the discarded plasma (saline or fresh frozen plasma, optionally with added human albumin). According to Ewald et al., its use was first reported in 1978, and it has since then been confirmed as a safe and reliable method for rapidly lowering excessive plasma TG levels, with a single session being capable of reducing TG levels by up to 70%. Filtration of plasma is also reported to be effective in reducing TG levels.

It would be desirable to develop improved treatments for severe hypertriglyceridemia, such as Type I or Type V hyperlipoproteinemia, especially for conditions that are refractory.

MBX-8025

MBX-8025 is the compound of the formula

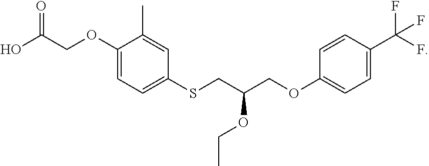 [SMILES: CCO[C@H](COc1ccc(C(F)(F)F)cc1)CSc1ccc(OCC(=O)O)c(C)c1]

MBX-8025 has the chemical name (R)-2-(4-((2-ethoxy-3-(4-(trifluoromethyl)phenoxy)propyl)thio)-2-methylphenoxy)acetic acid [IUPAC name as generated by CHEMDRAW ULTRA 12.0]. MBX-8025 and its synthesis, formulation, and use is disclosed in, for example, U.S. Pat. No. 7,301,050 (compound 15 in Table 1, Example M, claim 49), U.S. Pat. No. 7,635,718 (compound 15 in Table 1, Example M), and U.S. Pat. No. 8,106,095 (compound 15 in Table 1, Example M, claim 14). Lysine (L-lysine) salts of MBX-8025 and related compounds are disclosed in U.S. Pat. No. 7,709,682 (MBX-8025 L-lysine salt throughout the Examples, crystalline forms claimed).

MBX-8025 is an orally active, potent (2 nM) agonist of peroxisome proliferator-activated receptor-δ (PPARδ), It is specific (>600-fold and >2500-fold compared with PPARα and peroxisome proliferator-activated receptor-γreceptors). PPARδ activation stimulates fatty acid oxidation and utilization, improves plasma lipid and lipoprotein metabolism, glucose utilization, and mitochondrial respiration, and preserves stem cell homeostasis. According to U.S. Pat. No. 7,301,050, PPARδ agonists, such as MBX-8025, are suggested to treat PPARδ-mediated conditions, including “diabetes, cardiovascular diseases, Metabolic X syndrome, hypercholesterolemia, hypo-HDL-cholesterolemia, hyper-LDL-cholesterolemia, dyslipidemia, atherosclerosis, and obesity”, with dyslipidemia said to include hypertriglyceridemia and mixed hyperlipidemia.

A Phase 2 study of MBX-8025 L-lysine dihydrate salt in mixed dyslipidemia (6 groups, 30 subjects/group: once daily placebo, atorvastatin (ATV) 20 mg, or MBX-8025 L-lysine dihydrate salt at 50 or 100 mg (calculated as the free acid) capsules alone or combined with ATV 20 mg, for 8 weeks) has been reported by Bays et al., “MBX-8025, A Novel Peroxisome Proliferator Receptor-δ Agonist: Lipid and Other Metabolic Effects in Dyslipidemic Overweight Patients Treated with and without Atorvastatin”, J. Clin. Endocrin. Metab., 96(9), 2889-2897 (2011) and Choi et al., “Effects of the PPAR-δ agonist MBX-8025 on atherogenic dyslipidemia”, Atherosclerosis, 220, 470-476 (2012). Compared to placebo, MBX-8025 alone and in combination with ATV significantly (P<0.05) reduced apolipoprotein B-100 by 20-38%, LDL by 18-43%, TGs by 26-30%, non-HDL-C by 18-41%, free fatty acids by 16-28%, and high-sensitivity C-reactive protein by 43-72%; it raised HDL-C by 1-12% and also reduced the number of patients with the metabolic syndrome and a preponderance of small LDL particles. While MBX-8025 at 50 mg/day and 100 mg/day reduced TGs by 32% over the total population treated, the percentage reduction in TGs increased from near zero in the tertile of subjects with the lowest starting TG levels (125-155 mg/dL) to over 40% in the tertile with the highest starting TG levels (279-324 mg/dL). MBX-8025 corrects all three lipid abnormalities in mixed dyslipidemia—lowers TGs and LDL and raises HDL, selectively depletes small dense LDL particles, reduces cardiovascular inflammation, and improves other metabolic parameters including reducing serum aminotransferases, increases insulin sensitivity (lowers homeostatic model assessment-insulin resistance, fasting plasma glucose, and insulin), lowers GGT and ALP, significantly (>2-fold) reduces the percentage of subjects meeting the criteria for metabolic syndrome, and trends towards a decrease in waist circumference and increase in lean body mass. MBX-8025 was safe and generally well-tolerated, and also reduced liver enzyme levels.

The disclosures of the documents referred to in this application are incorporated into this application by reference.

### SUMMARY OF THE INVENTION

This invention is the treatment of severe hypertriglyceridemia, such as Type I or Type V hyperlipoproteinemia, for example conditions that are refractory, comprising therapy with MBX-8025 or an MBX-8025 salt, alone or in combination with one or more of a fibrate, niacin, and an omega-3 fatty acid; optionally accompanied by apheresis.

Because the effect of MBX-8025 on TG reduction has been seen to increase in dyslipidemic patients with higher starting TG levels, therapy with MBX-8025 or an MBX-8025 salt, alone or in combination with one or more of a fibrate, niacin, and an omega-3 fatty acid is expected to be especially effective where starting TG levels may be extremely elevated, as in severe hypertriglyceridemia.

This invention is a method of treating severe hypertriglyceridemia by administering MBX-8025 or an MBX-8025 salt, alone or in combination with one or more of a fibrate, niacin, and an omega-3 fatty acid.
Optional apheresis is also included.

Preferred embodiments of this invention are characterized by the specification and by the features of claims 1 to 20 of this application as filed.

### DETAILED DESCRIPTION OF THE INVENTION

Definitions

“Severe hypertriglyceridemia” and its treatment are described in paragraphs [0003] through [0015]. “Severe hypertriglyceridemia” refers to a serum TG level of ≧500 mg/dL, such as ≧750 mg/dL, for example ≧1000 mg/dL.

“Type I hyperlipoproteinemia” and “Type V hyperlipoproteinemia” are described especially in paragraph [0005].

“Refractory” is described in paragraph [0013].

“Fibrates” are described in paragraph [0010].

“Niacin” is described in paragraph [0011].

“Omega-3 fatty acids” are described in paragraph [0012].

“Apheresis” is described in paragraph [0015].

“MBX-8025” is described in paragraphs [0017] through [0020].

Salts (for example, pharmaceutically acceptable salts) of MBX-8025 are included in this invention and are useful in the compositions, methods, and uses described in this application. These salts are preferably formed with pharmaceutically acceptable acids. See, for example, “Handbook of Pharmaceutically Acceptable Salts”, Stahl and Wermuth, eds., Verlag Helvetica Chimica Acta, Zürich, Switzerland, for an extensive discussion of pharmaceutical salts, their selection, preparation, and use. Unless the context requires otherwise, reference to MBX-8025 is a reference both to the compound and to its salts.

Because MBX-8025 contains a carboxyl group, it may form salts when the acidic proton present reacts with inorganic or organic bases. Typically the MBX-8025 is treated with an excess of an alkaline reagent, such as hydroxide, carbonate or alkoxide, containing an appropriate cation. Cations such as Na⁺, K⁺, Ca²⁺, Mg²⁺, and NH₄⁺ are examples of cations present in pharmaceutically acceptable salts. Suitable inorganic bases, therefore, include calcium hydroxide, potassium hydroxide, sodium carbonate and sodium hydroxide. Salts may also be prepared using organic bases, such as salts of primary, secondary and tertiary amines, substituted amines including naturally-occurring substituted amines, and cyclic amines including isopropylamine, trimethylamine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, N-ethylpiperidine, and the like. As noted in paragraph [0020], MBX-8025 is currently formulated as its L-lysine dihydrate salt; and MBX-8025 has also been studied in clinical trials as its calcium salt.

“Combination therapy” with MBX-8025 and one or more of a fibrate, niacin, and an omega-3 fatty acid means the administration of MBX-8025 and a fibrate, niacin, an omega-3 fatty acid, or two or three of these additional agents during the course of treatment of SHTG. Such combination therapy may involve the administration of the MBX-8025 before, during, and/or after the administration of the fibrate, niacin, and an omega-3 fatty acid, such that therapeutically effective levels of each of the compounds are maintained. Because MBX-8025 is administered orally once/day, it may be convenient to administer MBX-8025 at the same time as the administration of the fibrate, niacin, and omega-3 fatty acid (if it or they are also administered once/day), or at the same time as one administration of the fibrate, niacin, and omega-3 fatty acid (if it or they are administered more than once/day). “Combination therapy” also includes the administration of a single dosage form (e.g. a capsule or tablet) containing both MBX-8025 and a fibrate and/or niacin: since omega-3 fatty acids are liquid, they will be administered separately, but may be provided in a treatment kit.

A “therapeutically effective amount” of MBX-8025 or an MBX-8025 salt means that amount which, when administered to a human for treating SHTG, is sufficient to effect treatment for SHTG. “Treating” or “treatment” of SHTG in a human includes one or more of:
(1) preventing or reducing the risk of developing SHTG, i.e., causing the clinical symptoms of SHTG, such as acute pancreatitis, not to develop in a subject who may be predisposed to SHTG but who does not yet experience or display symptoms of SHTG (i.e. prophylaxis);
(2) inhibiting SHTG, i.e., arresting or reducing the development of SHTG or its clinical symptoms; and
(3) relieving SHTG, i.e., causing regression, reversal, or amelioration of SHTG or reducing the number, frequency, duration or severity of its clinical symptoms.
The therapeutically effective amount for a particular subject varies depending upon the age, health and physical condition of the subject to be treated, the extent of the SHTG, the assessment of the medical situation, and other relevant factors. It is expected that the therapeutically effective amount will fall in a relatively broad range that can be determined through routine trial.

A “therapeutically effective amount” of each of (MBX-8025 or an MBX-8025 salt) and one or more of a fibrate, niacin, and omega-3 fatty acid means that amount of each compound which, when administered in combination therapy to a human for treating SHTG, is sufficient to effect treatment (as defined in paragraph [0038] above) of SHTG.

“Comprising” or “containing” and their grammatical variants are words of inclusion and not of limitation and mean to specify the presence of stated components, groups, steps, and the like but not to exclude the presence or addition of other components, groups, steps, and the like. Thus “comprising” does not mean “consisting of”, “consisting substantially of”, or “consisting only of”; and, for example, a formulation “comprising” a compound must contain that compound but also may contain other active ingredients and/or excipients.

Formulation and Administration

The MBX-8025 may be administered by any route suitable to the subject being treated and the nature of the subject's condition. Routes of administration include administration by injection, including intravenous, intraperitoneal, intramuscular, and subcutaneous injection, by transmucosal or transdermal delivery, through topical applications, nasal spray, suppository and the like or may be administered orally. Formulations may optionally be liposomal formulations, emulsions, formulations designed to administer the drug across mucosal membranes or transdermal formulations. Suitable formulations for each of these methods of administration may be found, for example, in “Remington: The Science and Practice of Pharmacy”, 20th ed., Gennaro, ed., Lippincott Williams & Wilkins, Philadelphia, Pa., U.S.A. Because MBX-8025 is orally available, typical formulations will be oral, and typical dosage forms will be tablets or capsules for oral administration. As mentioned in paragraph [0020], MBX-8025 has been formulated in capsules for clinical trials.

Depending on the intended mode of administration, the pharmaceutical compositions may be in the form of solid, semi-solid or liquid dosage forms, preferably in unit dosage form suitable for single administration of a precise dosage. In addition to an effective amount of the MBX-8025 (and optionally the fibrate and/or niacin), the compositions may contain suitable pharmaceutically-acceptable excipients, including adjuvants which facilitate processing of the active compounds into preparations which can be used pharmaceutically. “Pharmaceutically acceptable excipient” refers to an excipient or mixture of excipients which does not interfere with the effectiveness of the biological activity of the active compound(s) and which is not toxic or otherwise undesirable to the subject to which it is administered.

For solid compositions, conventional excipients include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by dissolving, dispersing, etc., an active compound as described herein and optional pharmaceutical adjuvants in water or an aqueous excipient, such as, for example, water, saline, aqueous dextrose, and the like, to form a solution or suspension. If desired, the pharmaceutical composition to be administered may also contain minor amounts of nontoxic auxiliary excipients such as wetting or emulsifying agents, pH buffering agents and the like, for example, sodium acetate, sorbitan monolaurate, triethanolamine sodium acetate, triethanolamine oleate, etc.

For oral administration, the composition will generally take the form of a tablet or capsule; or, especially for pediatric use, it may be an aqueous or nonaqueous solution, suspension or syrup. Tablets and capsules are preferred oral administration forms. Tablets and capsules for oral use will generally include one or more commonly used excipients such as lactose and corn starch. Lubricating agents, such as magnesium stearate, are also typically added. When liquid suspensions are used, the active agent may be combined with emulsifying and suspending excipients. If desired, flavoring, coloring and/or sweetening agents may be added as well. Other optional excipients for incorporation into an oral formulation include preservatives, suspending agents, thickening agents, and the like.

Typically, a pharmaceutical composition of MBX-8025, or a kit comprising compositions of MBX-8025, is packaged in a container with a label, or instructions, or both, indicating use of the pharmaceutical composition or kit in the treatment of SHTG.

Typically, a pharmaceutical composition of the combination of MBX-8025 and a fibrate and/or niacin, or a kit comprising separate compositions of MBX-8025 and of one or more of a fibrate, niacin, and an omega-3 fatty acid, is packaged in a container with a label, or instructions, or both, indicating use of the pharmaceutical composition or kit in the treatment of SHTG.

A suitable amount of MBX-8025 (calculated as the free acid) for oral dosing will be 20-200 mg/day, preferably 50-200 mg/day, for an adult subject with SHTG, depending on the stage of the SHTG and factors such as hepatic and renal function. That is, a suitable amount of MBX-8025 for oral dosing for adults in SHTG, such as an adult with Type I or Type V hyperlipoproteinemia, especially when the condition is refractory, will be similar to the amounts employed in clinical trials. Suitable reductions in dose toward the lower end of the outer range above will be made for subjects who are children, depending on such additional factors as age and body mass.

Suitable amounts of fibrates vary with the particular drug: for gemfibrozil, the recommended dosing (LOPID US package insert) is 1200 mg/day, administered as two 600 mg doses each 30 minutes before morning and evening meals; for fenofibrate, the recommended dosing (TRICOR US package insert) is 48-145 mg/day, administered as a single daily dose without regard to meals; for choline fenofibrate, the recommended dosing (TRILIPIX US package insert) is 45-135 mg/day (when calculated as fenofibric acid), administered as a single daily dose without regard to meals; for bezafibrate, the recommended dosing (BEZALIP New Zealand Medsafe data sheet) is 600 mg/day, administered as three 200 mg doses with or after meals, or for the controlled-release (BEZALIP Retard) formulation, 400 mg/day as a single dose in the morning or evening with or after a meal; and for ciprofibrate, the recommended dosing (ciprofibrate X-PIL UK patient information leaflet) is 100 mg/day as a single dose. A suitable amount of immediate release niacin (NIACOR US package insert) is 1-6 g/day, typically as 1-2 g two or three times/day; while a suitable amount of extended-release niacin (NIASPAN US package insert) is 0.5-2 g/day, especially 1-2 g/day, administered as a single dose at bedtime with a low-fat snack. Suitable amounts of omega-3 fatty acids (especially EPA and/or DHA) are 2 g/day or 4 g/day (EPANOVA US package insert), or 4 g/day (LOVAZA and VASCEPA US package inserts).

A person of ordinary skill in the art of the treatment of SHTG will be able to ascertain a therapeutically effective amount of the MBX-8025 or an MBX-8025 salt and, if desired, one or more of a fibrate, niacin, and an omega-3 fatty acid, for a particular disease, stage of disease, and patient to achieve a therapeutically effective amount without undue experimentation and in reliance upon personal knowledge and the disclosure of this application Similarly, such a person will be able to ascertain the therapeutic appropriateness of apheresis.

### EXAMPLES

The study is a 12-week interventional, open label (single blind), dose-escalation study using adult subjects (for example 30, preferably with at least one-fourth the number having Type I hyperlipoproteinemia and at least one-fourth the number having Type V hyperlipoproteinemia) with severe hypertriglyceridemia (fasting TG levels of at least 1000 mg/dL), on stable therapy (fibrates, niacin, O3FA) or refractory to such therapy. Exclusion criteria include stage 3 or 4 heart failure, uncontrolled diabetes mellitus in the month before screening, use of corticosteroids in the month before screening, estrogen treatments (contraceptive or hormone replacement) unless on a stable dose in the two months before screening, a history of pancreatic disease during the six months before screening, and current apheresis treatments. Subjects are assessed for fasting TGs and other lipids at baseline. Subjects initially receive MBX-8025 or an MBX-8025 salt orally at 50 mg/day (when calculated as the free acid), as a single dose each day, for four weeks; and are again assessed for fasting TGs and other lipids. Subjects then receive MBX-8025 or an MBX-8025 salt orally at 100 mg/day (when calculated as the free acid), as a single dose each day, for four weeks; and are again assessed for fasting TGs and other lipids. Finally, subjects receive MBX-8025 or an MBX-8025 salt orally at 200 mg/day (when calculated as the free acid), as a single dose each day, for four weeks; and are again assessed for fasting TGs and other lipids. The endpoints for the study are the mean absolute and percentage reduction in fasting TGs; the percentage of subjects achieving fasting TG levels of lower than 800, 500, and 300 mg/dL; and the percentage of subjects achieving at least a 30%, 40%, 50%, 60%, and 70% reduction in fasting TGs from baseline. Subjects will show a reduction in fasting TGs from baseline at each of the measurement points, with the reduction increasing with dose; and treatment of subjects with SHTG by administering MBX-8025 or an MBX-8025 salt will significantly reduce the percentage of subjects at risk of acute pancreatitis and associated adverse events.

A second study, using the same dosing of MBX-8025 or an MBX-8025 salt, but accompanied by one or more of a fibrate, niacin, and an omega-3 fatty acid at appropriate clinical dosages (as in paragraph [0052]) when one or more of such additional agents were not previously used, will show similar but increased lowering of fasting TG levels and reduced risk of acute pancreatitis.

A third study, using the same dosing as either the first or second study, will include apheresis at the initiation of therapy.

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

1. A method of treating severe hypertriglyceridemia by administering MBX-8025 or an MBX-8025 salt, alone or in combination with one or more of a fibrate, niacin, and an omega-3 fatty acid.

2. The method of claim 1 where the MBX-8025 or an MBX-8025 salt is MBX-8025 L-lysine dihydrate salt.

3. The method of claim 1 where the dose of MBX-8025 or an MBX-8025 salt (when calculated as the free acid) is 20-200 mg/day, preferably.

4. The method of claim 1 where the MBX-8025 or an MBX-8025 salt is administered once/day.

5. The method of claim 1 where the MBX-8025 or an MBX-8025 salt is administered alone.

6. The method of claim 1 where the MBX-8025 or an MBX-8025 salt is administered in combination with a fibrate.

7. The method of claim 6 where the fibrate is gemfibrozil, fenofibrate, choline fenofibrate, bezafibrate, or ciprofibrate.

8. The method of claim 7 where the dose of the fibrate is: for gemfibrozil, 1200 mg/day; for fenofibrate, 48-145 mg/day; for choline fenofibrate, 45-135 mg/day (calculated as fenofibric acid); for bezafibrate, 600 mg/day for immediate release or 400 mg/day for controlled release; and for ciprofibrate, 100 mg/day.

9. The method of claim 1 where the MBX-8025 or an MBX-8025 salt is administered in combination with niacin.

10. The method of claim 9 where the dose of niacin is 1-6 g/day for immediate release, or 0.5-2 g/day for controlled release.

11. The method of claim 1 where the MBX-8025 or an MBX-8025 salt is administered in combination with an omega-3 fatty acid.

12. The method of claim 11 where the dose of the omega-3 fatty acid is 2-4 g/day.

13. The method of claim 1 where the MBX-8025 or an MBX-8025 salt is administered in combination with two or more of a fibrate, niacin, and an omega-3 fatty acid.

14. The method of claim 1 where the MBX-8025 or an MBX-8025 salt is administered in combination with three of a fibrate, niacin, and an omega-3 fatty acid.

15. The method of claim 1 which includes treatment by apheresis.

16. The method of claim 1 where severe hypertriglyceridemia refers to a serum triglyceride level of at least 500 mg/dL.

17. The method of claim 1 where the severe hypertriglyceridemia is Type I or Type V hyperlipoproteinemia.

18. The method of claim 17 where the severe hypertriglyceridemia is Type I hyperlipoproteinemia.

19. The method of claim 17 where the severe hypertriglyceridemia is Type V hyperlipoproteinemia.

20. The method of claim 1 where the severe hypertriglyceridemia is refractory.
